(19) United States
(12) Patent Application Publication
(10) Pub. No.: US 2004/0049859 A1
(43) Pub. Date: Mar. 18, 2004

(54) Enzyme composition for bleaching human keratinous fibres and bleaching method

(76) Inventors: Gerard Lang, Saint Prix (FR); Gregory Plos, Paris (FR)

Correspondence Address: Finnegan Henderson Farabow, Garrett & Dunner, 1300 I Street NW, Washington, DC, 20005, US

(21) Appl. No.: 10/333,327

(22) PCT Filed: Jun. 29, 2001
(86) PCT No.: PCT/FR01/02091

(30) Foreign Application Priority Data
Jul. 21, 2000 (FR) 00/09620

(51) Int. Cl. A61K 7/13

(52) U.S. Cl. 8/405

(57) ABSTRACT

The invention concerns a ready-to-use composition for bleaching human keratinous fibres previously dyed with direct dyes, in particular hair, comprising at least a 4-electron oxidoreductase enzyme, and at least an enzymatic mediator. The invention also concerns a bleaching method using said composition.

The invention relates to a ready-to-use composition for bleaching human keratin fibers that have been dyed beforehand with direct dyes, in particular the hair, comprising at least one enzyme of 4-electron oxidoreductase type, and at least one enzyme mediator, and also to the bleaching process using this composition.

It is known practice to dye human keratin fibers, and in particular the hair, with dye compositions containing direct dyes such as neutral, acidic or cationic nitrobenzene direct dyes, neutral, acidic or cationic azo direct dyes, neutral, acidic or cationic quinone and in particular anthraquinone direct dyes, neutral, acidic or cationic indigoid dyes, azine direct dyes, triarylmethane direct dyes, indoamine direct dyes and natural direct dyes.

The variety of molecules used makes it possible to obtain a wide range of colors.

However, for various reasons such as the desire to partially or totally modify the shade thus given to the hair or the desire to remove this coloration, there may be cause to wish to partially or totally destroy the direct dyes fixed on or in the hair.

This bleaching may be performed via processes using oxidizing or reducing systems. However, these various systems have the drawback of impairing the keratin fibers especially by making them more fragile. There is thus a genuine need to carry out a bleaching treatment under milder conditions.

In patent application EP-1 062 938, an enzyme of 4-electron oxidoreductase type was used as an agent for oxidizing oxidation bases in a ready-to-use composition for the oxidation dyeing of keratin fibers containing at least one oxidation base.

The Applicant has now discovered, entirely surprisingly and unexpectedly, that it is possible to partially or totally bleach human keratin fibers that have been dyed beforehand with direct dyes, and in particular the hair, using a composition comprising at least one enzyme of 4-electron oxidoreductase type, and at least one enzyme mediator. The bleaching result obtained is uniform and homogeneous without giving rise to any significant degradation of the keratin fibers.

This discovery is the basis of the present invention.

A first subject of the invention is thus a ready-to-use composition for bleaching human keratin fibers that have been dyed beforehand with at least one direct dye, in particular the hair, characterized in that it comprises at least one enzyme of 4-electron oxidoreductase type, and at least one enzyme mediator, said composition being free of oxidation base.

Said bleaching may be partial or total.

A subject of the invention is also a bleaching process for bleaching human keratin fibers that have been dyed beforehand with at least one direct dye, in particular the hair, using a ready-to-use bleaching composition as described above.

The term “enzyme mediator” means any compound capable of increasing the enzymatic activity of said 4-electron oxidoreductase.

For the purposes of the invention, the expression “ready-to-use composition” means a composition intended to be applied in unmodified form to the keratin fibers, i.e. it may be stored in unmodified form before use or may result from the extemporaneous mixing of two or more compositions, for example a composition containing at least one 4-electron oxidoreductase and another comprising at least one enzyme mediator.

According to the invention, the enzyme mediator may be chosen from the compounds of formula (I) below, and the possible tautomeric forms thereof:

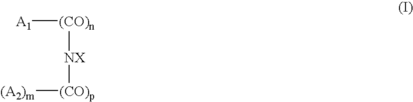

in which:

A₁ and A₂, which may be identical or different, represent:

a) a saturated or unsaturated, linear or branched aliphatic radical containing from 1 to 30 carbon atoms, it being possible for said aliphatic radical to be substituted with one or more hydroxyl, halo, sulfo, carboxyl, nitro or phenyl radicals;

b) a heterocyclic radical containing from 1 to 4 hetero atoms and from 5 to 10 ring members, it being possible for said heterocyclic radical to be substituted with one or more C₁-C₄ alkyl, halo, phenyl, hydroxyl or C₇-C₁₀ aralkyl radicals;

c) an aromatic radical comprising from 6 to 10 ring members, it being possible for said aromatic radical to be substituted with one or more C₁-C₄ alkyl, halo, sulfo, carboxyl, nitro, hydroxyl or nitroso radicals; it being possible for the nitrogen atom of the group NX to form with the groups A₁—(CO)ₙ and A₂—(CO)ₚ a heterocycle comprising from 5 to 18 ring members, it being possible for said heterocycle to be substituted with one or more C₁-C₄ alkyl, hydroxyl, phenyl, halo, sulfo, carboxyl or nitro radicals;

X represents a group —OH, ═O, ═S, →O or →S;

m, n and p, which may be identical or different, are integers equal to 0 or 1.

Among the enzyme mediators of formula (I) above, mention may be made in particular of hydroxylamine, N,N-dipropylhydroxylamine, N,N-diisopropylhydroxylamine, phenylhydroxylamine, N-acetylhydroxylamine, 1-phenyl-1H-1,2,3-triazole 1-oxide, 2,4,5-triphenyl-2H-1,2,3-triazole 1-oxide, 1-hydroxybenzotriazole, 1-hydroxybenzotriazolesulfonic acid, 1-hydroxybenzimidazole, N-hydroxyphthalimide, N-hydroxysuccinimide, quinoline N-oxide, isoquinoline N-oxide, 1-hydroxypiperidine, violuric acid, 4-hydroxy-3-nifrosocoumarin, 1,3-dimethyl-5-nitrosobarbituric acid, 1-nitroso-2-naphthol, 2-nitroso-1-naphthol-4-sulfonic acid, 2-nitroso-1-naphthol, 1-nitroso-2-naphthol-3,6-disulfonic acid and 2,4-dinitroso-1,3-dihydroxybenzene.

According to the invention, the enzyme mediator may also be chosen from the compounds of formula (II) or of formula (III) below:

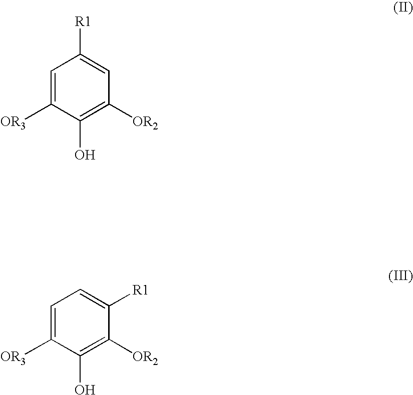

in which:

R₁ represents a group COR₄, CH═CHR₄, CH═CH—CH═CHR₄, CH═CHCOR₄, SO₂R₄ or POR₄R₅;

R₄ and R₅, independently of each other, denote a hydrogen atom, a hydroxyl radical, a C₁-C₅ alkyl radical, a C₁-C₅ alkoxy radical or a radical NR₆R₇;

R₆ and R₇, independently of each other, denote a hydrogen atom or a C₁-C₅ alkyl radical;

R₂ and R₃, independently of each other, denote a C₁-C₅ alkyl radical.

Among the enzyme mediators of formulae (II) and (III) above that may especially be mentioned are acetosyringone, syringaldehyde, methyl syringate, syringic acid, ethyl syringate, butyl syringate, hexyl syringate, octyl syringate or ethyl 3-(4-hydroxy-3,5-dimethoxyphenyl)acrylate.

According to the invention, the enzyme mediator may also be chosen from the compounds of formula (IV) below:

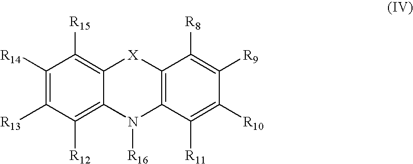

in which:

X represents a sulfur or oxygen atom;

R₈ to R₁₆, independently of each other, denote a hydrogen atom, a halogen atom, a hydroxyl, formyl, carboxyl, carboxyalkyl, carbamoyl, sulfo, sulfoalkyl, sulfamoyl, nitro, amino, phenyl, alkyl, alkoxy, carbonylalkyl or arylalkyl radical, these radicals possibly being substituted with one or more substituents R₁₇;

R₁₇ denotes a halogen atom or a hydroxyl, formyl, carboxyl, carboxyalkyl, carbamoyl, sulfo, sulfoalkyl, sulfamoyl, nitro, amino, phenyl, alkyl, aminoalkyl, piperidino, piperazinyl, pyrrolidino or alkoxy radical, these substituents themselves possibly being, where appropriate, substituted with one or more substituents R₁₇;

two of the substituents R₈ to R₁₆ possibly forming, together with the carbon atoms bearing them, a saturated or unsaturated ring optionally containing one or more hetero atoms, and optionally substituted with one or more substituents R₈.

Among the enzyme mediators of formula (IV) above that may especially be mentioned are 10-methylphenothiazine, 10-phenothiazinepropionic acid, N-hydroxysuccinimide-10-phenothiazine propionate, 10-ethyl-4-phenothiazinecarboxylic acid, 10-ethylphenothiazine, 10-propylphenothiazine, 10-isopropylphenothiazine, methyl-10-phenothiazinepropionate, 10-phenylphenothiazine, 10-allylphenothiazine, 10-[3-(4-methyl-1-piperazinyl)propyl]phenothiazine, 10-(2-pyrrolidinoethyl)phenothiazine, chlorpromazine, 2-chloro-10-methylphenothiazine, 2-acetyl-10-methylphenothiazine, 4-carboxy-10-phenothiazine, 10-methyl phenoxazine, 10-ethyl phenoxazine, 10-phenoxazinepropionic acid and 4-carboxy-10-phenoxazinepropionic acid.

2,2′-Azinobis(3-alkylbenzothiazoline-6-sulfonic acid) salts such as the diammonium salt of 2,2′-azinobis(3-ethylbenzothiazoline-6-sulfonic acid) may also be used as enzyme mediator.

The enzyme mediator(s) used in the composition in accordance with the invention preferably represent(s) from 0.0001% to 5% by weight approximately relative to the total weight of the composition, and preferably from 0.005% to 0.5% by weight approximately relative to this weight.

The 4-electron oxidoreductase(s) used in the composition in accordance with the invention can be chosen in particular from laccases, tyrosinases, catechol oxidases and polyphenol oxidases.

According to one specific and preferred embodiment of the invention, the 4-electron oxidoreductase(s) is (are) chosen from laccases.

These laccases can be chosen in particular from laccases of plant origin, of animal origin, of fungal origin (yeasts, molds and fungi) or of bacterial origin, the organisms of origin possibly being mono- or multicellular. The laccases can also be obtained by biotechnology.

Among the laccases of plant origin which can be used according to the invention, mention may be made of the laccases produced by plants which carry out chlorophyll synthesis, such as those mentioned in patent application FR-A-2 694 018.

Mention may be made in particular of the laccases present in extracts of Anacardiacea plants such as, for example, extracts of Magnifera indica, of Schinus molle or of Pleiogynium timoriense; in extracts of Podocarpacea plants; of Rosmarinus off.; of Solanum tuberosum; of Iris sp.; of Coffea sp.; of Daucus carrota; of Vinca minor; of Persea americana; of Catharanthus roseus; of Musa sp.; of Malus pumila; of Gingko biloba; of Monotropa hypopithys (Indian pipe), of Aesculus sp.; of Acer pseudoplatanus; of Prunus persica and of Pistacia palaestina.

Among the laccases of fungal origin, optionally obtained by biotechnology, which can be used according to the invention, mention may be made of the laccase(s) obtained from Polyporus versicolor, from Rhizoctonia praticola and from Rhus vernicifera as described, for example, in patent applications FR-A-2 112 549 and EP-A-504 005; the laccases described in patent applications WO 95/07988, WO 95/33836, WO 95/33837, WO 96/00290, WO 97/19998 and WO 97/19999, the content of which forms an integral part of the present description, such as, for example, the laccase(s) obtained from Scytalidium, from Polyporus pinsitus, from Myceliophthora thermophila, from Rhizoctonia solani, from Pyricularia orizae, and variants thereof. Mention may also be made of the laccase(s) obtained from Trametes versicolor, from Fomes fomentarius, from Chaetomium thermophile, from Neurospora crassa, from Colorius versicol, from Botrytis cinerea, from Rigidoporus lignosus, from Phellinus noxius, from Pleurotus ostreatus, from Aspergillus nidulans, from Podospora anserina, from Agaricus bisporus, from Ganoderma lucidum, from Glomerella cingulata, from Lactarius piperatus, from Russula delica, from Heterobasidion annosum, from Thelephora terrestris, from Cladosporium cladosporioides, from Cerrena unicolor, from Coriolus hirsutus, from Ceriporiopsis subvermispora, from Coprinus cinereus, from Panaeolus papilionaceus, from Panaeolus sphinctrinus, from Schizophyllum commune, from Dichomitius squalens, and from variants thereof.

Laccases of fungal origin, optionally obtained by biotechnology, will more preferably be chosen.

The enzymatic activity of the laccases used in accordance with the invention and having syringaldazine among their substrates can be defined by the oxidation of syringaldazine under aerobic conditions. One Lacu unit corresponds to the amount of enzyme which catalyzes the conversion of 1 mmol of syringaldazine per minute at a pH of 5.5 and at a temperature of 30° C. One U unit corresponds to the amount of enzyme which produces an absorbance delta of 0.001 per minute at a wavelength of 530 nm, using syringaldazine as substrate, at 30° C. and at a pH of 6.5. The enzymatic activity of the laccases used according to the invention can also be defined by the oxidation of para-phenylenediamine. One ulac unit corresponds to the amount of enzyme which produces an absorbance delta of 0.001 per minute at a wavelength of 496.5 nm, using para-phenylenediamine as substrate (64 mM), at 30° C. and at a pH of 5.

According to the invention, the enzymatic activity is preferably determined in ulac units.

In general, the 4-electron oxidoreductase(s) in accordance with the invention preferably represent(s) from 0.01% to 20% by weight approximately relative to the total weight of the composition, and even more preferably from 0.1% to 5% by weight approximately relative to this weight.

In particular, and when one or more laccases are used, the amount of laccase(s) present in the composition in accordance with the invention will vary as a function of the nature of the laccase(s) used. Preferably, the amount of laccase(s) is between 0.5 and 200 Lacu approximately (i.e. between 10,000 and 4×10⁶ U units approximately or alternatively between 20 and 2×10⁶ ulac units) per 100 g of composition.

The human keratin fibers that may be bleached according to the process of the invention are those dyed with at least one direct dye.

The direct dyes that may be used are preferably chosen from neutral, acidic or cationic nitrobenzene direct dyes, neutral, acidic or cationic azo direct dyes, neutral, acidic or cationic quinone and in particular anthraquinone direct dyes, neutral, acidic or cationic indigoid dyes, azine direct dyes, triarylmethane direct dyes, indoamine direct dyes and natural direct dyes.

Among the benzenic direct dyes that may be mentioned, in nonlimiting manner, are the following compounds:

1,4-diamino-2-nitrobenzene

1-amino-2-nitro-4-(β-hydroxyethylamino)benzene

1-amino-2-nitro-4-bis(β-hydroxyethyl)aminobenzene

1,4-bis(β-hydroxyethylamino)-2-nitrobenzene

1-β-hydroxyethylamino-2-nitro-4-bis-(β-hydroxyethylamino)benzene

1-β-hydroxyethylamino-2-nitro-4-aminobenzene

1-β-hydroxyethylamino-2-nitro-4-(ethyl)(β-hydroxyethyl)aminobenzene

1-amino-3-methyl-4-β-hydroxyethylamino-6-nitrobenzene

1-amino-2-nitro-4-β-hydroxyethylamino-5-chlorobenzene

1,2-diamino-4-nitrobenzene

1-amino-2-β-hydroxyethylamino-5-nitrobenzene

1,2-bis(β-hydroxyethylamino)-4-nitrobenzene

1-amino-2-[tris(hydroxymethyl)methylamino]-5-nitrobenzene

1-hydroxy-2-amino-5-nitrobenzene

1-hydroxy-2-amino-4-nitrobenzene

1-hydroxy-3-nitro-4-aminobenzene

1-hydroxy-2-amino-4,6-dinitrobenzene

1-β-hydroxyethyloxy-2-β-hydroxyethylamino-5-nitrobenzene

1-methoxy-2-β-hydroxyethylamino-5-nitrobenzene

1-β-hydroxyethyloxy-3-methylamino-4-nitrobenzene

1-β,γ-dihydroxypropyloxy-3-methylamino-4-nitrobenzene

1-β-hydroxyethylamino-4-β,γ-dihydroxypropyloxy-2-nitrobenzene

1-β,γ-dihydroxypropylamino-4-trifluoromethyl-2-nitrobenzene

1-β-hydroxyethylamino-4-trifluoromethyl-2-nitrobenzene

1-β-hydroxyethylamino-3-methyl-2-nitrobenzene

1-β-aminoethylamino-5-methoxy-2-nitrobenzene

1-hydroxy-2-chloro-6-ethylamino-4-nitrobenzene

1-hydroxy-2-chloro-6-amino-4-nitrobenzene

1-hydroxy-6-[bis(β-hydroxyethyl)amino]-3-nitrobenzene

1-β-hydroxyethylamino-2-nitrobenzene

1-hydroxy-4-β-hydroxyethylamino-3-nitrobenzene.

Among the azo direct dyes that may be mentioned are the cationic azo dyes described in patent applications WO 95/15144, WO 95/01772 and EP 714 954, the content of which forms an integral part of the invention.

Among the azo direct dyes that may also be mentioned are the following dyes described in the Color Index International 3rd edition:

Disperse Red 17; Acid Yellow 9; Acid Black 1; Basic Red 22; Basic Red 76; Basic Yellow 57; Basic Brown 16; Acid Yellow 36; Acid Orange 7; Acid Red 33; Acid Red 35; Basic Brown 17; Acid Yellow 23; Acid Orange 24; Disperse Black 9.

Mention may also be made of: 1-(4′-aminodiphenylazo)-2-methyl-4-[bis(β-hydroxyethyl)amino]benzene and 4-hydroxy-3-(2-methoxyphenylazo)-1-naphthalenesulfonic acid.

Among the quinone direct dyes that may be mentioned are the following dyes:

Disperse Red 15; Solvent Violet 13; Acid Violet 43; Disperse Violet 1; Disperse Violet 4; Disperse Blue 1; Disperse Violet 8; Disperse Blue 3; Disperse Red 11; Acid Blue 62; Disperse Blue 7; Basic Blue 22; Disperse Violet 15; Basic Blue 99,

and also the following compounds:

1-N-methylmorpholiniumpropylamino-4-hydroxyanthraquinone

1-aminopropylamino-4-methylaminoanthraquinone

1-aminopropylaminoanthraquinone

5-β-hydroxyethyl-1,4-diaminoanthraquinone

2-aminoethylaminoanthraquinone

1,4-bis(β,γ-dihydroxypropylamino)anthraquinone

Among the azine dyes that may be mentioned are the following compounds such as Basic Blue 17 and Basic Red 2.

Among the triarylmethane dyes that may be mentioned are the following compounds:

Basic Green 1; Acid Blue 9; Basic Violet 3; Basic Violet 14; Basic Blue 7; Acid Violet 49; Basic Blue 26; Acid Blue 7.

Among the indoamine dyes that may be mentioned are the following compounds:

2-β-hydroxyethylamino-5-[bis(4′-hydroxyethyl)amino]anilino-1,4-benzoquinone;

2-β-hydroxyethylamino-5-(2′-methoxy-4′-amino)anilino-1,4-benzoquinone;

3-N(2′-chloro-4′-hydroxy)phenylacetylamino-6-methoxy-1,4-benzoquinoneimine

3-N(2′-chloro-4′-methylamino)phenylureido-6-methyl-1,4-benzoquinoneimine;

3-[4′-N-(ethylcarbamylmethyl)amino]phenylureido-6-methyl-1,4-benzoquinoneimine.

Among the natural direct dyes that may be mentioned are lawsone, juglone, alizarin, purpurin, carminic acid, kermesic acid, purpurogallin, protocatechaldehyde, indigo, isatin, curcumin, spinulosin and apigenidin. Extracts or decoctions containing these natural dyes may also be used, and especially henna-based poultices or extracts.

Even more preferably, the bleaching process according to the invention will be used to totally or partially bleach hair that has been dyed with neutral, acidic or cationic nitrobenzene direct dyes, neutral or cationic azo direct dyes, cationic quinone and in particular anthraquinone direct dyes and indoamine direct dyes.

The medium that is suitable for bleaching (or support) for the ready-to-use bleaching composition in accordance with the invention generally consists of water or of a mixture of water and at least one organic solvent in order to dissolve the compounds which would not be sufficiently soluble in water. By way of organic solvent, mention may be made, for example, of C₁-C₄ alkanols such as ethanol and isopropanol; glycerol; glycols and glycol ethers such as 2-butoxyethanol; propylene glycol, propylene glycol monomethyl ether, diethylene glycol monoethyl ether and monomethyl ether, and aromatic alcohols such as benzyl alcohol or phenoxyethanol, similar products and mixtures thereof.

The solvents can be present in proportions preferably of between 1% and 40% by weight approximately relative to the total weight of the ready-to-use bleaching composition, and even more preferably between 5% and 30% by weight approximately.

The pH of the ready-to-use bleaching composition in accordance with the invention is chosen such that the enzymatic activity of the 4-electron oxidoreductase is sufficient. It is generally between 3 and 11 approximately, and preferably between 4 and 9 approximately. It may be adjusted to the desired value using acidifying or basifying agents usually used in the dyeing of keratin fibers.

Among the acidifying agents, mention may be made, by way of example, of inorganic or organic acids such as hydrochloric acid, orthophosphoric acid, sulfuric acid, carboxylic acids such as acetic acid, tartaric acid, citric acid or lactic acid, and sulfonic acids.

Among the basifying agents, mention may be made, by way of example, of aqueous ammonia, alkaline carbonates, alkanolamines such as mono-, di- and triethanolamines, 2-methyl-2-amino-1-propanol and derivatives thereof, sodium hydroxide, potassium hydroxide and the compounds of formula (V) below:

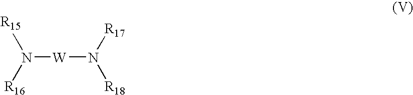

in which W is a propylene residue optionally substituted with a hydroxyl group or a C₁-C₄ alkyl radical; R₁₅, R₁₆, R₁₇ and R₁₈, which may be identical or different, represent a hydrogen atom or a C₁-C₄ alkyl or C₁-C₄ hydroxyalkyl radical.

The ready-to-use bleaching composition in accordance with the invention can also contain various adjuvants used conventionally in compositions for bleaching the hair, such as anionic, cationic, nonionic, amphoteric or zwitterionic surfactants or mixtures thereof, anionic, cationic, nonionic, amphoteric or zwitterionic polymers or mixtures thereof, mineral or organic thickeners, antioxidants, various enzymes of the 4-electron oxidoreductases used in accordance with the invention such as for example two electron oxidoreductases and/or peroxidases with the possible cofactors thereof, penetration agents, sequestering agents, fragrances, buffers, dispersants, conditioners such as, for example, volatile or nonvolatile, modified or unmodified silicones, film-forming agents, ceramids, preserving agents and opacifiers.

Needless to say, a person skilled in the art will take care to select this or these optional complementary compound(s) such that the advantageous properties intrinsically associated with the ready to use bleaching composition in accordance with the invention are not, or are not substantially, adversely affected by the envisioned addition(s).

The ready-to-use bleaching composition in accordance with the invention may be in various forms, such as in the form of liquids, creams or gels, which are optionally pressurized, or in any other form that is suitable for bleaching human keratin fibers, and especially the hair. When the composition is stored in unmodified form before use, it must be free of oxygen gas, so as to avoid any premature degradation of the mediator(s).

According to the bleaching process, at least one ready-to-use bleaching composition as defined above is applied to the fibers, at an application temperature of between room temperature and 80° C., for a period that is sufficient to partially or totally degrade the direct dyeing result on the human keratin fibers. Preferably, the fibers are then rinsed, or optionally washed with shampoo, and then dried.

The application temperature is preferably between room temperature and 60° C. and even more preferably between 35° C. and 50° C.

The time required to develop the bleaching result on the human keratin fibres is generally between 1 and 60 minutes and even more precisely between 5 and 30 minutes.

According to one specific embodiment of the invention, the process includes a first step which consists in separately storing, on the one hand, a composition (A) comprising, in a medium which is suitable for bleaching, at least one mediator as defined above, and, on the other hand, a composition (B) containing, in a medium which is suitable for bleaching, at least one enzyme of 4-electron oxidoreductase type, and then in mixing them together at the time of use, before applying this mixture to the keratin fibres.

Another subject of the invention is a multi-compartment bleaching device or “kit” according to the invention or any other multi-compartment packaging system, at least a first compartment of which contains composition (A) as defined above and at least a second compartment of which contains composition (B) as defined above. These devices can be equipped with means for applying the desired mixture to the hair, such as the devices described in patent FR-2 586 913 in the name of the Applicant.

The example that follows is intended to illustrate the invention without, however, limiting its scope.

### EXAMPLE

The ready-to-use bleaching composition below was prepared (contents in grams):

COMPOSITION

| | |
|---|---|
| 1-Hydroxybenzotriazole [enzyme mediator of formula (III)] | 0.1 |
| Laccase from Rhus vernicifera at 180 units/mg sold by the | 1.8 |
| company Sigma | |
| Bleaching support(*) | (*) |
| Demineralized water qs | 100 |

| | |
|---|---|
| (*): Bleaching support: | |
| Hydroxyethylcellulose sold under the trade name | |
| Natrosol 250 HHR ® by the company | |
| Aqualon | 1.0 g |
| 96° ethanol | 20.0 g |
| 2-Methyl-2-amino-1-propanol qs | pH 9.5 |

The ready-to-use bleaching composition described above was applied for 30 minutes at a temperature of 30° C. to locks of natural gray hair containing 90% white hairs which have been dyed beforehand with a solution containing 0.5% of 2-nitro-paraphenylenediamine in a mixture of water (90)/ethanol (10). The hair was then rinsed, washed with a standard shampoo and then dried.

The initial red shade was thus rendered considerably weaker.

1. A ready-to-use composition for bleaching human keratin fibers that have been dyed beforehand with at least one direct dye, in particular the hair, characterized in that it comprises at least one enzyme of 4-electron oxidoreductase type, and at least one enzyme mediator, said composition being free of oxidation base.

2. The composition as claimed in claim 1, characterized in that the enzyme mediator is chosen from the compounds of formula (I) below, and the tautomeric forms thereof: 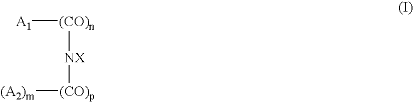
in which:
A₁ and A₂, which may be identical or different, represent:
a) a saturated or unsaturated, linear or branched aliphatic radical containing from 1 to 30 carbon atoms, it being possible for said aliphatic radical to be substituted with one or more hydroxyl, halo, sulfo, carboxyl, nitro or phenyl radicals;
b) a heterocyclic radical comprising from 1 to 4 hetero atoms and from 5 to 10 ring members it being possible for said heterocyclic radical to be substituted with one or more C₁-C₄ alkyl, halo, phenyl, hydroxyl or C₇-C₁₀ aralkyl radicals;
c) an aromatic radical comprising from 6 to 10 ring members, it being possible for said aromatic radical to be substituted with one or more C₁-C₄ alkyl, halo, sulfo, carboxyl, nitro, hydroxyl or nitroso radicals;
it being possible for the nitrogen atom of the group NX to form with the groups A₁—(CO)ₙ and A₂—(CO)ₚ a heterocycle comprising from 5 to 18 ring members, it being possible for said heterocycle to be substituted with one or more C₁-C₄ alkyl, hydroxyl, phenyl, halo, sulfo, carboxyl or nitro radicals;
X represents a group —OH, ═O, ═S, →O or →S;
m, n and p, which may be identical or different, are integers equal to 0 or 1.

3. The composition as claimed in claim 2, characterized in that the enzyme mediator(s) of formula (I) is (are) chosen from hydroxylamine, N,N-dipropylhydroxylamine, N,N-diisopropylhydroxylamine, phenylhydroxylamine, N-acetylhydroxylamine, 1-phenyl-1H-1,2,3-triazole 1-oxide, 2,4,5-triphenyl-2H-1,2,3-triazole 1-oxide, 1-hydroxybenzotriazole, 151-hydroxybenzotriazolesulfonic acid, 1-hydroxybenzimidazole, N-hydroxyphthalimide, N-hydroxysuccinimide, quinoline N-oxide, isoquinoline N-oxide, 1-hydroxypiperidine, violuric acid, 4-hydroxy-3-nitrosocoumarin, 1,3-dimethyl-5-nitrosobarbituric acid, 1-nitroso-2-naphthol, 2-nitroso-1-naphthol-4-sulfonic acid, 2-nitroso-1-naphthol, 1-nitroso-2-naphthol-3,6-disulfonic acid and 2,4-dinitroso-1,3-dihydroxybenzene.

4. The composition as claimed in claim 1, characterized in that the enzyme mediator is chosen from the compounds of formula (II) or of formula (III) below: 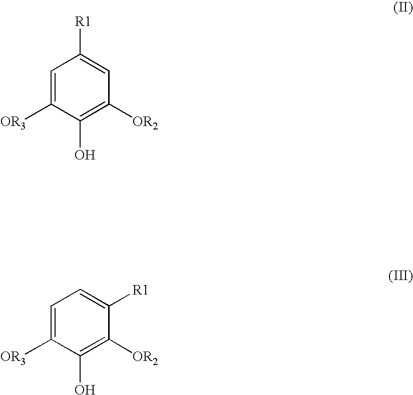
in which:
R₁ represents a group COR₄, CH═CHR₄, CH═CH—CH═CHR₄, CH═CHCOR₄, SO₂R₄ or POR₄R₅;
R₄ and R₅, independently of each other, denote a hydrogen atom, a hydroxyl radical, a C₁-C₅ alkyl radical, a C₁-C₅ alkoxy radical or a radical NR₆R₇;
R₆ and R₇, independently of each other, denote a hydrogen atom or a C₁-C₅ alkyl radical;
R₂ and R₃, independently of each other, denote a C₁-C₅ alkyl radical.

5. The composition as claimed in claim 4, characterized in that the enzyme mediator(s) of formulae (II) and (III) is (are) chosen from acetosyringone, syringaldehyde, methyl syringate, syringic acid, ethyl syringate, butyl syringate, hexyl syringate, octyl syringate or ethyl 3-(4-hydroxy-3,5-dimethoxyphenyl)acrylate.

6. The composition as claimed in claim 1, characterized in that the enzyme mediator is chosen from the compounds of formula (IV) below: 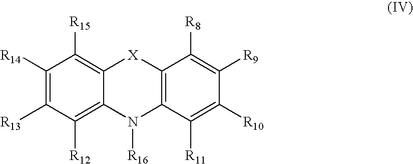
in which:
X represents a sulfur or oxygen atom;
R₈ to R₁₆, independently of each other, denote a hydrogen atom, a halogen atom, a hydroxyl, formyl, carboxyl, carboxyalkyl, carbamoyl, sulfo, sulfoalkyl, sulfamoyl, nitro, amino, phenyl, alkyl, alkoxy, carbonylalkyl or arylalkyl radical, these radicals possibly being substituted with one or more substituents R₁₇;
R₁₇ denotes a halogen atom or a hydroxyl, formyl, carboxyl, carboxyalkyl, carbamoyl, sulfo, sulfoalkyl, sulfamoyl, nitro, amino, phenyl, alkyl, aminoalkyl, piperidino, piperazinyl, pyrrolidino or alkoxy radical, these substituents themselves possibly being, where appropriate, substituted with one or more substituents R₁₇;
two of the substituents R₈ to R₁₆ possibly forming, together with the carbon atoms bearing them, a saturated or unsaturated ring optionally containing one or more hetero atoms, and optionally substituted with one or more substituents R₈.

7. The composition as claimed in claim 6, characterized in that the enzyme mediator(s) of formula (IV) above is (are) chosen from 10-methylphenothiazine, 10-phenothiazinepropionic acid, N-hydroxysuccinimide-10-phenothiazine propionate, 10-ethyl-4-phenothiazinecarboxylic acid, 10-ethylphenothiazine, 10-propylphenothiazine, 10-isopropylphenothiazine, methyl-10-phenothiazinepropionate, 10-phenylphenothiazine, 10-allylphenothiazine, 10-[3-(4-methyl-1-piperazinyl)propyl]phenothiazine, 10-(2-pyrrolidinoethyl)phenothiazine, chlorpromazine, 2-chloro-10-methylphenothiazine, 2-acetyl-10-methylphenothiazine, 4-carboxy-10-phenothiazine, 10-methyl phenoxazine, 10-ethyl phenoxazine, 10-phenoxazinepropionic acid and 4-carboxy-10-phenoxazinepropionic acid.

8. The composition as claimed in claim 1, characterized in that the enzyme mediator(s) is (are) chosen from 2,2′-azinobis(3-alkylbenzothiazoline-6-sulfonic acid) salts.

9. The composition as claimed in any one of the preceding claims, characterized in that the enzyme mediator(s) represent(s) 0.0001% to 5% by weight relative to the total weight of the composition and preferably 0.005% to 0.5% relative to this weight.

10. The composition as claimed in any one of the preceding claims, characterized in that the 4-electron oxidoreductase(s) is (are) chosen from laccases, tyrosinases, catechol oxidases and polyphenol oxidases.

11. The composition as claimed in claim 10, characterized in that the laccase(s) is (are) of plant origin, of animal origin, of fungal origin (yeasts, molds and fungi) or of bacterial origin, or is (are) obtained by biotechnology.

12. The composition as claimed in claim 11, characterized in that the laccase is of plant origin and is chosen from the laccases present in extracts of Anacardiacea plants; of Podocarpacea plants; of Rosmarinus off.; of Solanum tuberosum; of Iris sp.; of Coffea sp.; of Daucus carrota; of Vinca minor; of Persea americana; of Catharanthus roseus; of Musa sp.; of Malus pumila; of Gingko biloba; of Monotropa hypopithys (Indian pipe), of Aesculus sp.; of Acer pseudoplatanus; of Prunus persica and of Pistacia palaestina.

13. The composition as claimed in claim 11, characterized in that the laccase is of fungal origin or is obtained by biotechnology.

14. The composition as claimed in claim 13, characterized in that the laccase is chosen from the laccases obtained from Polyporus versicolor, from Rhizoctonia praticola, from Rhus vernicifera, from Scytalidium, from Polyporus pinsitus, from Myceliophthora thermophila, from Rhizoctonia solani, from Pyricularia orizae, from Trametes versicolor, from Fomes fomentarius, from Chaetomium thermophile, from Neurospora crassa, from Colorius versicol, from Botrytis cinerea, from Rigidoporus lignosus, from Phellinus noxius, from Pleurotus ostreatus, from Aspergillus nidulans, from Podospora anserina, from Agaricus bisporus, from Ganoderma lucidum, from Glomerella cingulata, from Lactarius piperatus, from Russula delica, from Heterobasidion annosum, from Thelephora terrestris, from Cladosporium cladosporioides, from Cerrena unicolor, from Coriolus hirsutus, from Ceriporiopsis subvermispora, from Coprinus cinereus, from Panaeolus papilionaceus, from Panaeolus sphinctrinus, from Schizophyllum commune, from Dichomitius squalens, and from variants thereof.

15. The composition as claimed in any one of the preceding claims, characterized in that the 4-electron oxidoreductases represent 0.01% to 20% by weight relative to the total weight of the composition.

16. The composition as claimed in claim 15, characterized in that the 4-electron oxidoreductase(s) represent(s) 0.1% to 5% by weight relative to the total weight of the composition.

17. The composition as claimed in any one of claims 10 to 14, characterized in that the amount of laccase(s) is between 0.5 and 200 Lacu per 100 g of composition.

18. The composition as claimed in any one of the preceding claims, characterized in that it has a pH of between 4 and 9.

19. The composition as claimed in any one of the preceding claims, characterized in that it also contains one or more adjuvants chosen from anionic, cationic, nonionic, amphoteric or zwitterionic surfactants or mixtures thereof, anionic, cationic, nonionic, amphoteric or zwitterionic polymers or mixtures thereof, mineral or organic thickeners, antioxidants, various enzymes of the 4-electron oxidoreductases according to the invention, penetration agents, sequestering agents, fragrances, buffers, dispersants, volatile or nonvolatile, modified or unmodified silicones, film-forming agents, ceramids preserving agents and opacifiers.

20. The composition as claimed in claim 19, characterized in that it also contains at least one peroxidase and/or one two-electron oxidoreductase and the possible cofactor thereof.

21. A process for bleaching human keratin fibers that have been dyed beforehand with at least one direct dye, and in particular the hair, characterized in that at least one composition as defined in any one of claims 1 to 20 is applied to said fibers, at an application temperature of between room temperature and 80° C., for a period that is sufficient to partially or totally degrade the coloration of the fibers.

22. The process as claimed in claim 21, characterized in that the application temperature is between 35° C. and 50° C.

23. The process as claimed in claim 21 or 22, characterized in that the time required to develop the bleaching result is between 1 and 60 minutes.

24. The process as claimed in claim 23, characterized in that the time required to develop the bleaching result is between 5 and 30 minutes.

25. The process as claimed in any one of claims 21 to 24, characterized in that the direct dye(s) is (are) chosen from neutral, acidic or cationic nitrobenzene direct dyes, neutral, acidic or cationic azo direct dyes, neutral, acidic or cationic quinone and in particular anthraquinone direct dyes, neutral, acidic or cationic indigoid dyes, azine direct dyes, triarylmethane direct dyes, indoamine direct dyes and natural direct dyes.

26. The process as claimed in claim 25, characterized in that the direct dye(s) is (are) chosen from neutral, acidic or cationic nitrobenzene direct dyes, neutral or cationic azo direct dyes, cationic quinone and in particular anthraquinone direct dyes and indoamine direct dyes.

27. The process as claimed in any one of claims 21 to 26, characterized in that it includes a first step which consists in separately storing, on the one hand, a composition (A) comprising, in a medium which is suitable for bleaching, at least one enzyme mediator as defined in any one of claims 2 to 8, and, on the other hand, a composition (B) containing, in a medium which is suitable for bleaching, at least one enzyme of 4-electron oxidoreductase type, and then in mixing them together at the time of use, before applying this mixture to the keratin fibres.

28. A multi-compartment device for bleaching human keratin fibers that have been dyed with at least one direct dye, and in particular the hair, characterized in that it comprises at least one first compartment containing composition (A) as defined in claim 27 and at least one second compartment containing composition (B) as defined in claim 27.
